(19) United States
(12) Patent Application Publication
(10) Pub. No.: US 2019/0330600 A1
(43) Pub. Date: Oct. 31, 2019

(54) GLUTATHIONE REDUCTASE

(71) Applicant: DSM IP Assets B.V., Heerlen (NL)

(72) Inventors: Marco Alexander VAN DEN BERG, Echt (NL); Elaheh JAMALZADEH, Echt (NL); Samuel Adrianus Maria RUINARD, Echt (NL)

(21) Appl. No.: 16/472,099

(22) PCT Filed: Dec. 14, 2017
(86) PCT No.: PCT/EP2017/082784

§ 371 Date: Jun. 20, 2019

(30) Foreign Application Priority Data
Dec. 22, 2016 (EP) 16206155.0

(51) Int. Cl. C12N 9/02

(52) U.S. Cl. CPC C12N 9/0051

(57) ABSTRACT

The present invention relates to a polypeptide having glutathione reductase activity selected from the group consisting of: a) polypeptide having an amino acid sequence comprising the mature polypeptide sequence of SEQ ID NO: 1 or a 2) a polypeptide comprising an amino acid sequence that has at least 50% sequence identity with the mature polypeptide sequence of SEQ ID NO: 1.

### FIELD OF THE INVENTION

The present invention relates to a polypeptide having glutathione reductase activity and to a composition comprising such a polypeptide. The invention also relates to a nucleic acid encoding an glutathione reductase, to an expression vector comprising the nucleic acid and to a recombinant host cell comprising the nucleic acid or expression vector. The invention further relates to a method for the preparation of the polypeptide and to use of the polypeptide for the reduction of cystine. The invention in addition relates to a method for the enzymatic reduction of cystine.

### BACKGROUND

Glutathione reductase (also abbreviated as GR) (EC 1.8.1.7) catalyzes the reduction of glutathione disulfide (GSSG) to the sulfhydryl form glutathione (GSH), which is a useful molecule in resisting oxidative stress and maintaining a reducing environment. Resisting oxidative stress and providing a reducing environment is beneficial in food applications, for example for the preservation of food items.

One application which could benefit from the reducing environment of GSH is the reduction of cystine, or L-cystine, towards cysteine, or L-cysteine. Cysteine is a precursor in the food, pharmaceutical and personal care industries. One of the larger food applications is the production of flavours. For example, the reaction of cysteine with sugars in a Maillard reaction yields meat flavours. Cysteine is also used as a processing aid for baking and is added to natural fruit juice products as antioxidant. When used as a food additive, cysteine has the E number E920.

Although variant glutathione reductases catalyse the same conversion, this does not mean that they are suitable for the same applications. Various applications will place different demands on the conditions under which the enzymes have to operate. Physical and chemical parameters that may influence the rate of an enzymatic conversion are the temperature (which has a positive effect on the reaction rates, but may have a negative effect on enzyme stability), the moisture content, the pH, the salt concentration, the structural integrity of the food matrix, the presence of activators or inhibitors of the enzyme, the concentration of the substrate and products, etc.

Therefore there exists an ongoing need for improved glutathione reductases for several applications having improved properties, for example in higher temperature applications where thermostability may be advantageous.

For the application in food items, it is important that the GSH is free of microbial contamination. One solution to avoid microbial contamination is to produce GSH at elevated temperature, such as at least 40° C.

### SUMMARY

The present invention is based on the identification of a polypeptide having glutathione reductase activity. The polypeptide may be derived from, for example, a microorganism of the genus Chaetomium such as from the species Chaetomium thermophilum.

A polypeptide of the invention is preferably thermophilic, for example thermostable (i.e. capable of withstanding a thermal treatment in respect of its enzymatic activity) and/or thermoactive (i.e. only develops its full enzymatic activity at elevated temperature). A polypeptide of the invention may alternatively or additionally be one which is active across a broad pH range and/or at a relatively high or low pH.

Providing a glutathione reductase with improved thermophilic properties is an important way of broadening its application. Thermoactive and thermostable glutathione reductase has substantial advantages over other glutathione reductase. For instance, the reduction of the GSSG can be conducted at comparatively high temperatures using thermoactive or thermostable glutathione reductase, and this results in a compatibility with processes in which high temperatures play a role. Moreover, the reduction of GSSG at higher temperatures can be conducted at a higher reaction rate.

Glutathione reductases active at a broad pH range are also advantageous since it may be possible to use a polypeptide of the invention in different processes with widely differing pH ranges. It is also possible to use such a polypeptide in processes in which the pH value is subject to significant fluctuations in the process. Processes are also possible in which pH values from 6 to 10 occur.

The polypeptide of the invention having glutathione reductase activity may be used, in particular, in the production of food ingredients, preferably in the reduction of cystine to cysteine at a temperature above 40° C. This is beneficial because it reduces the risk of microbial contamination of the food ingredient.

Accordingly, the present invention provides a polypeptide having glutathione reductase activity selected from the group consisting of:
a) a polypeptide having an amino acid sequence comprising the mature polypeptide sequence of SEQ ID NO: 1;
b) a polypeptide comprising an amino acid sequence that has at least 50% sequence identity with the mature polypeptide sequence of SEQ ID NO: 1;
c) a polypeptide encoded by a nucleic acid comprising a sequence that hybridizes under medium stringency conditions to the complementary strand of the mature polypeptide encoding sequence of SEQ ID NO: 2; and
d) a polypeptide comprising an amino acid sequence encoded by a nucleic acid that has at least 50% sequence identity to the mature polypeptide coding sequence of SEQ ID NO: 2

The invention also provides:
A nucleic acid encoding a polypeptide having glutathione reductase activity according to claim 1 or claim 2, which comprises a sequence that has at least 50% sequence identity to the mature polypeptide encoding sequence of SEQ ID NO: 2.
An expression vector comprising a nucleic acid according to claim 3, operably linked to one or more control sequences that direct expression of the glutathione reductase in a host cell.
An recombinant host cell comprising a polypeptide of the invention, a nucleic acid of the invention or an expression vector of the invention.
A method for the preparation of a polypeptide of the invention, which method comprises:
   cultivating a host cell according of the invention in a suitable fermentation medium under conditions that allow for production of the polypeptide; and, optionally,
   recovering the polypeptide.
Use of a polypeptide of the invention, for the reduction of oxidized thiols.
A method for the enzymatic reduction of cystine to cysteine, which method comprises:
   contacting cystine with a reduction solution comprising a polypeptide of the invention, a cofactor and a mediator; and, optionally, recovering the cysteine.
A composition comprising cysteine and a polypeptide of the invention.

### BRIEF DESCRIPTION OF THE DRAWINGS

FIG. 1 sets out the expression vector as used in example 1.

FIG. 2 sets out the temperature activity of the fungal glutathione reductase polypeptide from Chaetomium thermophilum and yeast glutathione reductase at different temperatures.

FIG. 3 sets out the relative activity of the glutathione reductase polypeptide from Chaetomium thermophilum and yeast glutathione reductase at different pHs.

FIG. 4 sets out residual activity of the fungal glutathione reductase polypeptide from Chaetomium thermophilum and yeast glutathione reductase at different temperatures after 30 minutes.

FIG. 5 sets out the formation of cysteine using glutathione reductase polypeptide from Chaetomium thermophilum and yeast glutathione reductase.

### DESCRIPTION OF THE SEQUENCE LISTING

SEQ ID NO: 1 sets out the amino acid sequence of a glutathione reductase polypeptide from Chaetomium thermophilum.

SEQ ID NO: 2 sets out the nucleotide sequence encoding the amino acid sequence of a glutathione reductase polypeptide from Chaetomium thermophilum, codon-pair optimized for expression in Saccharomyces cerevisiae.

### DETAILED DESCRIPTION

The term “complementary strand” can be used interchangeably with the term “complement”. The complementary strand of a nucleic acid can be the complement of a coding strand or the complement of a non-coding strand. When referring to double-stranded nucleic acids, the complement of a nucleic acid encoding a polypeptide refers to the complementary strand of the strand encoding the amino acid sequence or to any nucleic acid molecule containing the same. Typically, the reverse complementary strand is intended.

The term “expression” includes any step involved in the production of the polypeptide including, but not limited to, transcription, post transcriptional modification, translation, post-translational modification, and secretion.

An “expression vector” comprises a polynucleotide coding for a polypeptide, operably linked to the appropriate control sequences (such as a promoter, and transcriptional and translational stop signals) for expression and/or translation in vitro, or in the host cell of the polynucleotide.

The expression vector may be any vector (e.g., a plasmid or virus), which can be conveniently subjected to recombinant DNA procedures and can bring about the expression of the polynucleotide. The choice of the vector will typically depend on the compatibility of the vector with the cell into which the vector is to be introduced. The vectors may be linear or closed circular plasmids. The vector may be an autonomously replicating vector, i.e. a vector, which exists as an extra-chromosomal entity, the replication of which is independent of chromosomal replication, e.g., a plasmid, an extra-chromosomal element, a mini-chromosome, or an artificial chromosome. Alternatively, the vector may be one which, when introduced into the host cell, is integrated into the genome and replicated together with the chromosome(s) into which it has been integrated. The integrative cloning vector may integrate at random or at a predetermined target locus in the chromosomes of the host cell. The vector system may be a single vector or plasmid or two or more vectors or plasmids, which together contain the total DNA to be introduced into the genome of the host cell, or a transposon.

A “host cell” as defined herein is an organism suitable for genetic manipulation and one which may be cultured at cell densities useful for industrial production of a target product, such as a polypeptide according to the present invention. A host cell may be a host cell found in nature or a host cell derived from a parent host cell after genetic manipulation or classical mutagenesis. Advantageously, a host cell is a recombinant host cell.

The term “hybridization” means the pairing of substantially complementary strands of oligomeric compounds, such as nucleic acid compounds.

Hybridization may be performed under low, medium or high stringency conditions. Low stringency hybridization conditions comprise hybridizing in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency hybridization conditions comprise hybridizing in 6×SSC at about 45° C., followed by one or more washes in 0.2×SSC, 0.1% SDS at 60° C., and high stringency hybridization conditions comprise hybridizing in 6×SSC at about 45° C., followed by one or more washes in 0.2×SSC, 0.1% SDS at 65° C.

A nucleic acid or polynucleotide sequence is defined herein as a nucleotide polymer comprising at least 5 nucleotide or nucleic acid units. A nucleotide or nucleic acid refers to RNA and DNA. The terms “nucleic acid” and “polynucleotide sequence” are used interchangeably herein.

The term “polypeptide” refers to a molecule comprising amino acid residues linked by peptide bonds and containing more than five amino acid residues. The term “protein” as used herein is synonymous with the term “polypeptide” and may also refer to two or more polypeptides. Thus, the terms “protein” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity in the presence of a specific substrate under certain conditions may be referred to as enzymes. It will be understood that, as a result of the degeneracy of the genetic code, a multitude of nucleotide sequences encoding a given polypeptide may be produced.

An “isolated nucleic acid fragment” is a nucleic acid fragment that is not naturally occurring as a fragment and would not be found in the natural state.

The term “isolated polypeptide” as used herein means a polypeptide that is removed from at least one component, e.g. other polypeptide material, with which it is naturally associated. The isolated polypeptide may be free of any other impurities. The isolated polypeptide may be at least 50% pure, e.g., at least 60% pure, at least 70% pure, at least 75% pure, at least 80% pure, at least 85% pure, at least 80% pure, at least 90% pure, or at least 95% pure, 96%, 97%, 98%, 99%, 99.5%, 99.9% as determined by SDS-PAGE or any other analytical method suitable for this purpose and known to the person skilled in the art. An isolated polypeptide may be produced by a recombinant host cell.

Preferably the present polypeptide, or composition comprising the present polypeptide, is free from other enzyme activities than glutathione reductase enzyme activity. More preferably the present polypeptide is, or composition comprising the present polypeptide, is free from peptidase activity, protease activity and/or phosphatase activity.

A “mature polypeptide” is defined herein as a polypeptide in its final form and is obtained after translation of a mRNA into polypeptide and post-translational modifications of said polypeptide. Post-translational modification include N-terminal processing, C-terminal truncation, glycosylation, phosphorylation and removal of leader sequences such as signal peptides, propeptides and/or prepropeptides by cleavage.

A “mature polypeptide coding sequence” means a polynucleotide that encodes a mature polypeptide (with reference to its amino acid sequence).

The term “nucleic acid construct” is herein referred to as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term “expression cassette” when the nucleic acid construct contains all the control sequences required for expression of a coding sequence, wherein said control sequences are operably linked to said coding sequence.

The term “promoter” is defined herein as a DNA sequence that is bound by RNA polymerase and directs the polymerase to the correct downstream transcriptional start site of a nucleic acid sequence to initiate transcription. A promoter may also comprise binding sites for regulators.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all. The term “recombinant” is synonymous with “genetically modified” and “transgenic”.

The terms “sequence identity” or “sequence homology” are used interchangeably herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleotides/bases or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See the homepage of the National Center for Biotechnology Information at http://www.ncbi.nlm.nih.gov/.

A “synthetic molecule”, such as a synthetic nucleic acid or a synthetic polypeptide is produced by in vitro chemical or enzymatic synthesis. It includes, but is not limited to, variant nucleic acids made with optimal codon usage for host organisms of choice.

A synthetic nucleic acid may be optimized for codon use, preferably according to the methods described in WO2006/077258 and/or WO2008000632, which are herein incorporated by reference. WO2008/000632 addresses codon-pair optimization. Codon-pair optimization is a method wherein the nucleotide sequences encoding a polypeptide that have been modified with respect to their codon-usage, in particular the codon-pairs that are used, are optimized to obtain improved expression of the nucleotide sequence encoding the polypeptide and/or improved production of the encoded polypeptide. Codon pairs are defined as a set of two subsequent triplets (codons) in a coding sequence. Those skilled in the art will know that the codon usage needs to be adapted depending on the host species, possibly resulting in variants with significant homology deviation from SEQ ID NO: 2, but still encoding the polypeptide according to the invention.

The present invention relates to a polypeptide having glutathione reductase activity (EC 1.8.1.7) selected from the group consisting of:
a) a polypeptide having an amino acid sequence comprising the mature polypeptide sequence of SEQ ID NO: 1, or having an amino acid sequence comprising the polypeptide sequence of SEQ ID NO: 1;
b) a polypeptide comprising an amino acid sequence that has at least 50% sequence identity with the mature polypeptide sequence of SEQ ID NO: 1, or has at least 50% sequence identity with the polypeptide sequence of SEQ ID NO: 1;
c) a polypeptide encoded by a nucleic acid comprising a sequence that hybridizes under medium stringency conditions to the complementary strand of the mature polypeptide encoding sequence of SEQ ID NO: 2 or of the polypeptide encoding sequence of SEQ ID NO 2; and
d) a polypeptide comprising an amino acid sequence encoded by a nucleic acid that has at least 50% sequence identity to the mature polypeptide coding sequence of SEQ ID NO: 2 or to the polypeptide coding sequence of SEQ ID NO: 2.

The advantage of the present polypeptide is that it is heat stable and retains its activity at higher temperatures. This allows the use of the present polypeptide in for example food processes comprising a heat treatment to reduce contamination, without inactivating the activity of the polypeptide.

The invention also provides a polypeptide of the invention which is:

i. a polypeptide comprising an amino acid sequence that has at least 60%, 70%, 75%, 80%, 85%, 90%, 91%, 92%, 93%, 94%, 95%, 96%, 97%, 98% or 99% sequence identity with the (mature) polypeptide sequence of SEQ ID NO: 1;

ii. a polypeptide encoded by a nucleic acid comprising a sequence that hybridizes under high stringency conditions to the complementary strand of the mature polypeptide encoding sequence of SEQ ID NO: 2 (or the corresponding wild-type sequence or a sequence codon optimized or codon pair optimized for expression in a heterologous organism, such as a yeast for example Saccharomyces cerevisiae); or

ii. a polypeptide comprising an amino acid sequence encoded by a nucleic acid that has at least 60%, 70%, 75%, 80%, 85%, 90%, 91%, 92%, 93%, 94%, 95%, 96%, 97%, 98% or 99% sequence identity to the (mature) polypeptide coding sequence of SEQ ID NO: 2 (or the corresponding wild-type sequence or a sequence codon optimized or codon pair optimized for expression in a heterologous organism, such as a yeast for example Saccharomyces cerevisiae).

The invention also relates to polypeptides which are isolated, substantially pure, pure, recombinant, synthetic or variant polypeptides of such polypeptides.

A polypeptide of the invention may be derivable from an organism of the genus Chaetomium, such as from Chaetomium thermophilum. The wording “derived” or “derivable” from with respect to the origin of a polypeptide of the invention means that when carrying out a BLAST search with a polypeptide according to the present invention, the polypeptide according to the present invention may be derivable from a natural source, such as a microbial cell, of which an endogenous polypeptide shows the highest percentage homology or identity with the polypeptide as disclosed herein.

Preferably, a polypeptide of the invention may be a polypeptide that has least 50%, 60%, 70%, 75%, 80%, 85, 86%, 87%, 88%, 89% 90%, 91%, 92%, 93%, 94%, 95%, 96%, 97%, 98%, or at least 99% or 100% sequence identity to the (mature) polypeptide sequence of SEQ ID NO: 1.

A polypeptide according to the present invention may be encoded by any suitable polynucleotide sequence. Typically a polynucleotide sequence is codon optimized, or a codon pair optimized sequence for expression of a polypeptide as disclosed herein in a particular host cell. A polypeptide of the invention may be encoded by a polynucleotide sequence that comprises appropriate control sequences and/or signal sequences, for example for secretion.

A polypeptide of the invention may be encoded by a polynucleotide that hybridizes under medium stringency, preferably under high stringency conditions to the complementary strand of the mature polypeptide coding sequence of SEQ ID NO: 2 (or the corresponding wild-type sequence or a sequence codon optimized or codon pair optimized for expression in a heterologous organism, such as a Bacillus, for example Bacillus subtilis).

A polypeptide of the invention may also be encoded by a nucleic acid that has at least 80%, 85%, 86%, 87%, 88%, 89% 90%, 91%, 92%, 93%, 94%, 95%, 96%, 97%, 98%, or at least 99% or 100% identity to a (mature) polypeptide coding sequence of SEQ ID NO: 2 (or the corresponding wild-type sequence or a sequence codon optimized or codon pair optimized for expression in a heterologous organism, such as a yeast for example Saccharomyces cerevisiae).

A polypeptide of the invention may also be a variant of a mature polypeptide of SEQ ID NO: 1, comprising a substitution, deletion and/or insertion at one or more positions of the mature polypeptide SEQ ID NO: 1. A variant of the mature polypeptide of SEQ ID NO: 1 may be an amino acid sequence that differs in 1, 2, 3, 4, 5, 6, 7, 8, 9, 10, 11 or 12 or more than 12 amino acids from the amino acids of the mature polypeptide of SEQ ID NO: 1.

In one embodiment the present invention features a biologically active fragment of a polypeptide as disclosed herein.

A polypeptide of the invention may be a naturally occurring polypeptide or a genetically modified or recombinant polypeptide.

A polypeptide of the invention may be purified. Purification of proteins is known to a skilled person in the art.

A polypeptide of the invention may preferably be thermostable and/or thermoactive. Additionally or alternatively, a polypeptide of the invention may be active across a broad pH range and/or active at a relatively high or low pH.

A polypeptide of the invention may be thermostable. “Thermostable” herein means that a polypeptide of the invention may have a residual glutathione reductase activity of at least 60% after an incubation period of 30 min at 50° C. A polypeptide of the invention may have a residual glutathione reductase activity of at least 50% after an incubation period of 30 min at 55° C., 60° C., 65° C. or 70° C.

“Residual activity” herein means any specific/volumetric enzymatic activity that an enzyme has after a specific incubation duration at a specific temperature compared with the original specific/volumetric activity in the range of its temperature optimum under otherwise identical reaction conditions (pH, substrate etc.). The specific/volumetric activity of an enzyme means a specific amount of a converted substrate (for example in pmol) per unit time (for example in minutes) per enzyme amount (for example in mg or ml). The residual activity of an enzyme results from the specific/volumetric activity of the enzyme after the aforementioned incubation duration divided by the original specific/volumetric activity expressed as a percentage (%). In this case, the specific activity of an enzyme may be indicated in U/mg and the volumetric activity of an enzyme may be indicated in U/ml. Alternatively, the specific/volumetric activity of an enzyme can also be indicated in katal/mg or katal/ml in the sense of the description.

The term “enzymatic activity”, sometimes also referred to as “catalytic activity” or “catalytic efficiency”, is generally known to the person skilled in the art and refers to the conversion rate of an enzyme and is usually expressed by means of the ratio kₖₐₜ/K_{M}, wherein kₖₐₜ is the catalytic constant (also referred to as turnover number) and the K_{M} value corresponds to the substrate concentration, at which the reaction rate lies at half its maximum value. Alternatively, the enzymatic activity of an enzyme can also be specified by the specific activity (μmol of converted substrate×mg⁻¹×min⁻¹; cf. above) or the volumetric activity (μmol of converted substrate×ml⁻¹×min⁻¹; cf. above).

Reference can also be made to the general literature such as Structure and Mechanism in Protein Science: A guide to enzyme catalysis and protein folding, Alan Fersht, W.H.Freeman, 1999; Fundamentals of Enzyme Kinetics, Athel Cornish-Bowden, Wiley-Blackwell 2012 and Voet et al., “Biochemie” [Biochemistry], 1992, VCH-Verlag, Chapter 13, pages 331-332 with respect to enzymatic activity.

Thus, a thermostable polypeptide of the invention may have a residual activity of at least 60%, at least 70%, at least 80%, at least 90% after 30 minutes at a temperature of 50° C. Preferably, a polypeptide of the invention has a residual activity in the range of from 75% to 100%, such as 75% to 90% after 30 minutes at a temperature of 50° C.

A polypeptide of the invention having glutathione reductase activity is preferably thermoactive. “Thermoactive” herein means that the temperature optimum of such a polypeptide is at least about 50° C., at least about 55° C., at least about 60° C.

Thermoactivity may be determined as set out in Example 2.

The term “temperature optimum” is generally known to the skilled person and relates to the temperature range at which an enzyme exhibits its maximum enzymatic activity. Reference can be made in association with this to the relevant literature such as Enzyme Assays: A Practical Approach, Robert Eisenthal, Michael J. Danson, Oxford University Press 2002; Voet et al., “Biochemie”, 1992, VCH-Verlag, Chapter 13, page 331; I. H. Segel, Enzyme Kinetics: Behavior and Analysis of Rapid Equilibrium and Steady-State Enzyme Systems, Wiley Interscience, 1993; and A. G. Marangoni, Enzyme Kinetics: A Modern Approach, Wiley Interscience, 2002.

Herein, the temperature optimum is preferably understood to be the temperature range, in which a polypeptide of the invention has at least 80%, preferably at least 90% of the maximum enzymatic activity under otherwise constant reaction conditions.

The temperature optimum of a polypeptide according to the invention preferably lies in the range of from 50° to 70° C., such as in the range of from 55′ to 65° C.

Thus, a polypeptide of the invention is preferably thermostable (i.e. capable of withstanding a thermal treatment in respect of its enzymatic activity) and/or thermoactive (i.e. only develops its full enzymatic activity at elevated temperature).

A polypeptide of the invention may be active at a relatively high pH, such as a pH of at least 6, at least 7, at least 8 or at least 9.

The term “pH optimum” is generally known to the skilled person and relates to the pH range, in which an enzyme has its maximum enzymatic activity. Reference can be made in association with this to the relevant literature such as Enzyme Assays: A Practical Approach, Robert Eisenthal, Michael J. Danson, Oxford University Press 2002 and Voet et al., “Biochemie”, 1992, VCH-Verlag, Chapter 13, page 331. Herein, the term pH optimum is typically understood to mean the pH range, in which the glutathione reductase used according to the invention has at least 80%, preferably at least 90% of the maximum enzymatic activity under otherwise constant reaction conditions.

A polypeptide of the invention may be active over a very broad pH range. In the range from pH 6 to pH 10, a polypeptide of the invention may preferably have an activity of at least 10% of the maximum activity. As a result of this, it may possible to use a polypeptide of the invention in different processes with widely differing pH ranges. It is also possible to use it in processes in which the pH value is subject to significant fluctuations in the process. Processes are also possible in which pH values from 6 to 10 occur.

Over the entire pH range of from pH 6.0 to pH 10, a polypeptide of the invention has an activity of at least 10%, more preferred at least 15%, further preferred at least 20%, most preferred at least 25% and in particular at least 30% compared to the maximum activity, i.e. to the maximum activity with the optimum pH value under otherwise identical conditions, preferably at optimum temperature and concentration.

The invention further provides a nucleic acid encoding a polypeptide having glutathione reductase activity, which comprises a sequence that at least 50% sequence identity to the mature polypeptide encoding sequence of SEQ ID NO: 2.

A nucleic acid of the invention may comprise a polynucleotide sequence encoding a polypeptide of the invention which has at least 60%, 70%, 75%, 80%, 85%, 86%, 87%, 88%, 89% 90%, 91%, 92%, 93%, 94%, 95%, 96%, 97%, 98%, or 99% sequence identity to SEQ ID NO: 2, or to the mature polypeptide coding sequence of either thereof.

A polynucleotide sequence of the invention may comprise SEQ ID NO: 2 or may comprise the mature polypeptide coding sequence of either thereof.

A nucleic acid of the invention may be an isolated, substantially pure, pure, recombinant, synthetic or variant nucleic acid of the nucleic acid of SEQ ID NO: 2. A variant nucleic acid sequence may for instance have at least 80% sequence identity to SEQ ID NO: 2.

The invention also provides a nucleic acid construct comprising a nucleic acid of the invention. An expression vector which comprises a nucleic acid of the invention or a nucleic acid of the invention operably linked to one or more control sequences that direct expression of the polypeptide in a host cell.

There are several ways of inserting a nucleic acid into a nucleic acid construct or an expression vector which are known to a skilled person in the art, see for instance Sambrook & Russell, Molecular Cloning: A Laboratory Manual, 3rd Ed., CSHL Press, Cold Spring Harbor, N.Y., 2001. It may be desirable to manipulate a nucleic acid encoding a polypeptide of the present invention with control sequences, such as promoter and terminator sequences.

A promoter may be any appropriate promoter sequence suitable for a eukaryotic or prokaryotic host cell, which shows transcriptional activity, including mutant, truncated, and hybrid promoters, and may be obtained from polynucleotides encoding extracellular or intracellular polypeptides either endogenous (native) or heterologous (foreign) to the cell. The promoter may be a constitutive or inducible promoter. An inducible promoter may be, for example, a starch inducible promoter.

The present invention also provides a host cell, or recombinant host cell, comprising a nucleic acid or an expression vector as disclosed herein. A suitable host cell may be a mammalian, insect, plant, fungal, or algal cell, or a bacterial cell.

Preferably, the host cell, or recombinant host cell, is a eukaryote, more preferably a yeast. More preferably, the present host cell is Saccharomyces cerevisiae or recombinant Saccharomyces cerevisiae.

More preferably, the present host cell is a thermolable host cell, or a mesophilic host cell, or a host cell with activity peak below 35° C. The advantage of a thermolable host cell is that undesired enzymatic side activities can be easily reduced by heat treatment at for example 40 to 75° C.

Preferably, the present recombinant host cell, preferably being Saccharomyces cerevisiae, further comprises a nucleic acid encoding a dehydrogenase enzyme, preferably a thermostable and/or thermoactive dehydrogenase enzyme. Preferably a nucleic acid encoding a glucose dehydrogenase enzyme which is derived from Bacillus, preferably derived from Bacillus megaterium or Bacillus subtilis. Examples of an enzyme derived from Bacillus subtilis are known from U.S. Pat. Nos. 5,126,256 and 5,114,853. More preferably the gene construct allows an over expression of dehydrogenase enzyme, such as glucose dehydrogenase enzyme. The advantage of combining a nucleic acid encoding a dehydrogenase enzyme with the nucleic acid or an expression vector as disclosed herein, is that the recombinant host cells provides both enzymes. The (glucose) dehydrogenase enzyme can advantageously be used for regeneration of cofactors.

The present inventors found that by using Saccharomyces cerevisiae as host, undesired side activities such as peptidases, proteases and phosphatases are reduced.

The invention also relates to a method for the production of a polypeptide of the invention comprising cultivating a host cell in a suitable fermentation medium under conditions conducive to the production of the polypeptide and producing the polypeptide. A skilled person in the art understands how to perform a process for the production of a polypeptide as disclosed herein depending on a host cell used, such as pH, temperature and composition of a fermentation medium. Host cells can be cultivated in shake flasks, or in fermenters having a volume of 0.5 or 1 litre or larger to 10 to 100 or more cubic metres. Cultivation may be performed aerobically or anaerobically depending on the requirements of a host cell.

Advantageously a polypeptide as disclosed herein is recovered or isolated from the fermentation medium.

Alternatively the polypeptide as disclosed herein is not recovered from the isolated fermentation medium. The fermentation medium, or the present recombinant host cell, or Saccharomyces cerevisiae can immediately be used for the enzymatic reduction of for the reduction of oxidized thiols, without being recovered from the fermentation medium.

Therefore, the present invention relates to the use of the present polypeptide for the reduction of oxidized thiols. Preferably, the present invention relates to the use of the present polypeptide for the reduction of oxidized glutathione or for the production of reduced glutathione. More preferably, the present invention relates to the use of the present polypeptide for the reduction of oxidized thiols with a mediator, preferably glutathione. More preferably the present invention relates to the use of the present polypeptide for the reduction of cystine. More preferably, the present invention relates to the use of the present polypeptide for the reduction of oxidized thiols at a temperature within the range of 25° C. to 75° C., preferably at a temperature within the range of 30° C. to 65° C., or 35° C. to 60° C., more preferably within the range of 40° C. to 75° C., most preferably within the range of 45° C. to 75° C. or even 50° C. or 60° C. to 75° C.

The present invention also relates to a method for the enzymatic reduction of cystine to cysteine, which method comprises:
contacting cystine with a reduction solution comprising the polypeptide of the invention, a cofactor and a mediator; and, optionally,
recovering the cysteine.

The present inventors found that the polypeptide of the invention could efficiently be used for the enzymatic reduction of cystine. The present polypeptide particularly allows an enzymatic reduction of cystine at elevated temperatures, which is beneficial in reduction the risk of microbial contamination of the cysteine. This is advantageous in food applications of the cysteine.

Therefore, in a preferred embodiment, the present step of contacting the cystine with the reduction solution is carried out at a temperature within the range of 25° C. to 75° C., preferably at a temperature within the range of 30° C. to 65° C., or 35° C. to 60° C., more preferably within the range of 40° C. to 75° C., most preferably within the range of 45° C. to 75° C. or even 50° C. or 60° C. to 75° C.

A cofactor, as used in the present context, is intended to mean a helper molecule that assists the polypeptide of the invention. Preferably, the cofactor is a coenzyme. Preferably, the cofactor is nicotinamide adenine dinucleotide phosphate (NADPH), preferably in oxidized form (NADP+) or nicotinamide adenine dinucleotide (NADH), preferably in oxidized form (NAD+).

A mediator as used in the present context is intended to mean compounds which facilitate the electron transfer process, i.e. compounds that catalyze the transfer of electrons to cystine leading to reduced cysteine. Preferably, the present mediator is glutathione. Glutathione might be in oxidized form (also abbreviated as GSSG) or in reduced form (also abbreviated as GSH).

In a preferred embodiment, the present reduction solution has a pH of at least 6. More preferably, the present reduction solution has a pH of at least 6.5, at least 7, at least 7.5, at least 8.5 or at least 9.0. More preferably, the pH of the reduction solution is at most 7, at most 8, at most 9, at most 10, at most 11 or at most 12.

In a preferred embodiment, the present reduction solution further comprises a cofactor regeneration system. The use of a cofactor regeneration system provides an improved process for the enzymatic reduction of cystine since no extra step is necessary for addition of cofactor during the course of the reduction. Furthermore, it is cost efficient to regenerate cofactor since lower amounts of cofactor are needed to provide the reduction of cystine to cysteine. Preferably the cofactor regeneration system comprises an enzyme and a corresponding substrate. Preferably the cofactor regeneration system comprises glucose dehydrogenase and glucose and/or formate dehydrogenase and formate. Preferably the glucose dehydrogenase and glucose and/or formate dehydrogenase is a thermostable and/or thermoactive enzyme. More preferably the glucose dehydrogenase is derived from Bacillus, more preferably derived from Bacillus megaterium or Bacillus subtilis. Thermostable dehydrogenase enzymes are known from for example U.S. Pat. Nos. 5,126,256 and 5,114,853 and describe dehydrogenase derived from B megaterium. Formate dehydrogenase is found particularly suitable for the regeneration of NADH, whereas glucose dehydrogenase is suitable for regeneration of both NADPH and NADH. Alternative enzymes in the present cofactor regeneration system are alcohol dehydrogenase, an NADP-dependent formate dehydrogenase, glucose 6-phosphate dehydrogenase, H2-driven NAD(P)+-reducing hydrogenase or phosphite dehydrogenase. Particularly preferred is a cofactor regeneration system comprising alcohol dehydrogenase and an alcohol, more preferably alcohol dehydrogenase with isopropanol or alcohol dehydrogenase with ethanol. The advantage of using alcohol dehydrogenase with an alcohol as substrate is that the products formed by the alcohol dehydrogenase, such as acetone after using isopropanol and acetaldehyde after using ethanol, are volatile and therefore they could be easily removed from the present reduction solution and/or cysteine comprising solution.

In a preferred embodiment the method of the invention is carried out on industrial scale. Throughout the description of the invention, an industrial scale method or an industrial process may be understood to encompass a method using a reduction solution having a volume scale which is ≥10 L, preferably ≥100 L, more preferably ≥1 m³, ≥5 m³, even more preferably ≥10 m³, most preferably ≥25 m³, preferably less than 250 m³.

In another preferred embodiment, the present reduction contains low, i.e. less than 5% (wt) or less than 1% (wt) or no compounds that can catalyze the reoxidation of cysteine to cystine. Examples of such compounds that have a tendency to ‘adsorp’ electrons, are ferric iron, nitrate, cupric ions. The advantage is an increased amount of cysteine in the present reduction solution.

In yet another preferred embodiment the present reduction solution is an aqueous solution. Preferably the reduction solution or aqueous solution comprises a buffer. Preferably the buffer is a sodium phosphate buffer. Alternatively, the buffer is a tris(hydroxymethyl)aminomethane buffer (or tris HCl buffer) or a 2-(N-morpholino)ethanesulfonic acid buffer (abbreviated as MES buffer).

Given the advantage that the cysteine produced by the present enzymatic reduction is free of microbial contamination, and thus allows a safe use in food application, the present invention relates to a composition comprising cysteine and the polypeptide of the invention. More specifically the present composition is a food composition. A food composition is a composition which is regarded as safe and/or which is suitable for use in the manufacturing of food items. More preferably, the present composition further comprises one or more selected from gluconic acid, formic acid, glucose, formate, nicotinamide adenine dinucleotide phosphate, nicotinamide adenine dinucleotide, glutathione (GSH or GSSG), acetone and acetaldehyde.

Furthermore, the cysteine resulting from the present invention can advantageously be used in food items, in food matrices or for the production of process flavours.

The invention is further illustrated in the non-limiting examples below.

### EXAMPLES

### Example 1: Expression of a Glutathione Reductase from Chaetomium thermophilum in S. cerevisiae

A polypeptide having glutathione reductase (GR) activity that functions at high temperature, was obtained from Chaetomium thermophilum, originating from Chaetomium thermophilum var. thermophilum, strain DSM 1495.

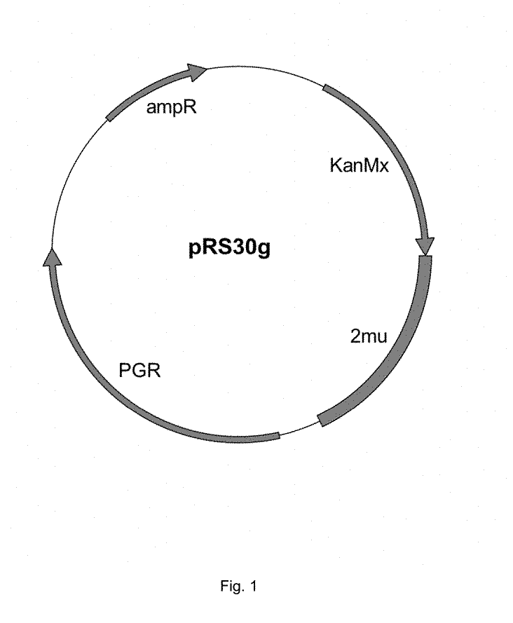

The amino acid sequence of SEQ ID NO: 1 was used as template for S. cerevisiae specific codon pair optimization, as described in WO2008/000632. For cloning purposes a DNA sequence containing Bsal recognition sites was introduced at the 5′- and 3′-end of the gene encoding fungal GR. By using golden gate cloning the fungal GR gene was placed in between the S. cerevisiae TDH3 promoter and TAL1 terminator and subsequently transformed to E. coli for propagation. Transformants were selected using the kanamycin resistance gene which confers resistance against Neomycin. Plasmids containing the THD3 promoter-fungal GR SEQ ID NO: 2-TAL1 terminator (POT) expression cassettes were isolated and used as template in a PCR reaction for amplification of the POT and simultaneous addition of 50 base pair DNA sequences homologous to 5′- and 3′-ends of linearized S. cerevisiae 2-micron expression vector pRS30g as shown in FIG. 1. The fungal GR POT expression cassettes and linearized pRS30g were co-transformed via a PEG lithium acetate (LIAC) method to the publicly available S. cerevisiae CEN.PK113-7D lacking endogenous glutathione reductase activity (ΔGLR1). The vector backbone and fungal GR POT recombine in vivo via homologous recombination into a functional expression vector. Selection of transformants was done using the KanMx marker present on pRS30g which confers G418 resistance. Correct transformants were cultivated in a yeast extract, phytone, D glucose medium (YEPh-D medium)+G418 (200 μg/ml). This pre culture was used to inoculate the fermentation in Verduyn media (Verduyn C, Postma E, Scheffers W A, van Dijken J P. 1992a. Effect of Benzoic Acid on Metabolic Fluxes in Yeasts: A Continuous-Culture Study on the Regulation of Respiration and Alcoholic Fermentation. Yeast 8:501-517.)+G418 (400 ml in 2 L shakefiask, 200 RPM, 30° C.) to a start OD600 of 0.1. After 24 hours of incubation, cells were centrifuged, washed with milli-Q water (mQ) and suspended in 200 mM Tris-HCL buffer (pH 7.5) to 10% wlw based on wet pellet weight. Suspension was transferred in one ml portions to two ml tubes containing 500 mg glassbeads (0.45-0.50 mm). Cells were mechanically lysed by thoroughly shaking the tubes three-times at 5000 RPM for 40 seconds on the Precellys 24 homogenizer with two minutes of cooling on ice in between. Insoluble cell debris was removed by centrifugation at 4° C. for 15 minutes at 12.000 RPM, pooling of supernatants and centrifugation for 60 minutes at 3000 RPM. This cell free extract was used as source of glutathione reductase.

### Example 2: Biochemical Characterization of the Thermostable Glutathione Reductase

### 2.1 Temperature Dependent Activity of Thermostable Fungal Glutathione Reductase & Yeast Glutathione Reductase

The activity of the enzyme according to example 1 and a commercial available yeast glutathione reductase from Saccharomyces cerevisiae (Sigma-Aldrich, G3664) enzymes at different temperatures was quantified by incubation of the enzyme with 10 mM GSSG and 0.5 mM NADPH in the assay buffer 0.1 M Tris pH 8.0 for 15 min and measuring the absorbance at 340 nm immediately afterwards. The reactions were incubated in glass test tubes in a water bath set to the desired temperature (approximately every 10° C. between 20 and 80° C.) in a dilution that was expected to yield an acceptable conversion of NADPH in 15 min at that temperature. After 15 min, the reactions were quenched by rapid cooling on melting ice and the readout was performed within 2 minutes. One tube was added as a blank (buffer added instead of enzyme) and an additional blank was not incubated in the water bath, but stored on ice during the reaction (ice blank). The ice blank was measured twice, once at the start and once at the end of the day. The measured absorbance was corrected for the corresponding blank (incubated at the same temperature) and the different dilution factors and activities were expressed relative to the highest observed activity for the enzyme. The composition of the reaction mixture at the start of the reaction was as follows: 250 microliter assay buffer pH 8.0+250 microL 40 mM GSSG+250 microL 2 mM NADPH+250 microliter enzyme dilution to start the reaction.

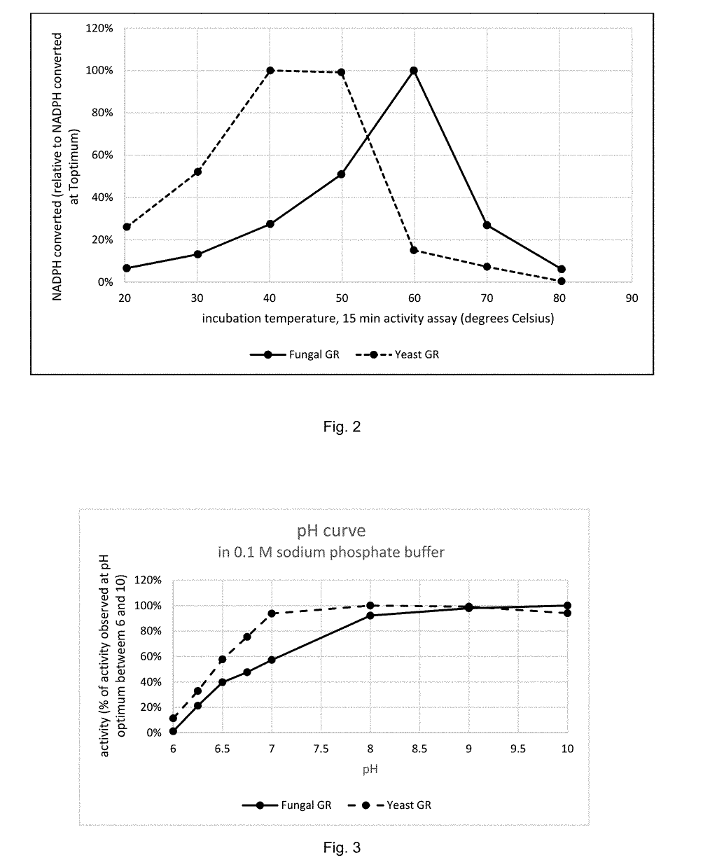

The relative activities of GR are depicted in FIG. 2. The fungal thermostable glutathione reductase shows maximal catalytic activity at 60° C.

### 2.2 pH Dependent Activity of Fungal Thermostable Glutathione Reductase

The pH curve for both GR enzymes was obtained by assaying the activity at every pH unit between pH 6 and 10 in 0.1 M phosphate buffer at room temperature. A blank without GR enzyme was included at every pH. The buffers for pH 6-9 were made by mixing of 0.1 M Na2HPO4 solution and 0.1M NaH2PO4 solution at different ratio's to obtain the desired pH. Addition of sodium hydroxide was required for reaching a buffer pH 10. An NADPH stock of 50 mM was prepared in milliQ water with added sodium hydroxide to pH 8, where NADPH is soluble and stable. The GSSG solutions were prepared separately for each pH (20 mM=61.3 mg/5 mL buffer in 15 mL Greiner tube). The enzymes were first diluted 20× in water and then 100× in buffer at the different pH set points. The NADPH stock solution was diluted 25× in the desired buffer briefly before starting the assay to minimize the background conversion of NADPH before addition of enzyme. The assay was performed as follows: 100 microL GSSG solution pH X+50 microliter NADPH solution pH X+50 microliter diluted enzyme pH X, next kinetic readout 340 nm at desired pH was carried out. The slopes of the reactions (Δ340 nm/min) were corrected for the slope of the blank reaction (buffer added instead of enzyme) at the same pH and activities were expressed relative to the highest measured activity for that enzyme. The first measuring series was for the fungal GR (non-heat shocked), the second series was for the yeast GR.

The relative activity is shown in FIG. 3. The fungal GR shows maximal catalytic activity at pH between 8 and 10.

### 2.3 Thermal Stability of Fungal Glutathione Reductase (30 Minutes)

The fungal GR produced in example 1 was heat shocked to deactivate possible protease activities. The sample was diluted 100-fold in buffer to prevent any stabilizing effect of glycerol in the sample solution and the sample was subjected to 65° C. for 60 min in a water bath. The sample was diluted to pre-equilibrated buffer (0.1 M Tris pH 8.0) in a 15 mL plastic Greiner tube. The tube was then closed and placed back in the water bath immediately. After 60 min the tube was put on melting ice for efficient cooling. The sample was stored refrigerated. The heat shocked fungal GR (already 100×dil) and commercial yeast GR (Sigma-Aldrich G3664) were diluted 20× and 2500× respectively, in the assay buffer (0.1 M Tris pH 8.0). The solutions were transferred to the wells of 2 PCR plates (100 microL per well, 3 columns of 8 wells per enzyme sample); the remaining dilutions were kept on melting ice. The PCR equipment was programmed to apply a temperature gradient across the plate in the direction of the columns. One PCR plate was incubated at temperatures ranging from 64 to 40° C. (from A→H=64/62.5/59.4/54.6/49.2/44.6/41.5/40° C.), the other at temperatures ranging from 56 to 80° C. (from A→H=80/78.5175.4170.6/65.2/60.6/57.5/56° C.). The temperature gradient was held for 30 min and then the PCR plates were rapidly cooled (in the PCR equipment) back to a constant temperature of 4° C. until analysis (for ˜20 min in this case). Both plates were incubated in the PCR equipment at the same time but in different machines (of the same type).

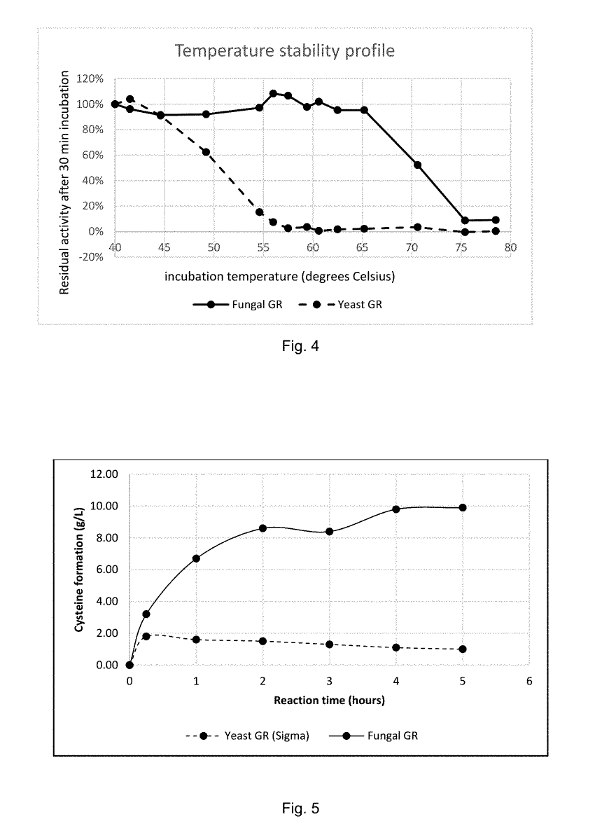

Temperature stability profiles for both GR enzymes are shown in FIG. 4. The Fungal GR is stable at temperature ranges between 40 to 65 degrees while yeast GR loses more than 80% of its activity after 30 minutes incubation at 55 degrees.

### 2.4 Thermal Stability of Fungal Glutathione Reductase in Application (16 Hours)

The heat shocked fungal GR (already 100×dil) and commercial yeast GR (Sigma-Aldrich G3664) were diluted 20× and 2500× in buffer (0.1 M Tris pH 8). The samples were transferred to the wells of 2 PCR plates (100 microL per well); the remaining dilutions were stored refrigerated overnight. One plate was incubated at 30° C. and the other at 50° C. in a PCR machine for 16 hours (lid temp 105° C.). After 16 hours the PCR program brought the temperature back down to 4° C. until the activity analysis. For the activity analysis, 50 microliter was transferred from the wells of the PCR plate to the GR activity assay in MTP. A blank (buffer instead of enzyme) was included for correction of the slope (Δ340 nm/min) with the background conversion of NADPH. The results of the different wells (8 per enzyme per temperature) were averaged and expressed relative to the activity of the sample stored overnight in the fridge (=100%, assuming the enzyme is stable under those conditions). The 16-hour incubations of the heat-shocked fungal GR and the commercially available yeast GR yielded the following result (table 1):

TABLE 1
overview of the results of the overnight incubation of the GR
enzymes at (slightly) elevated temperatures

| | Residual activity after | Residual activity after |
|---|---|---|
| Enzyme | 16 hrs at 30° C. | 16 hrs at 50° C. |
| Yeast GR (Sigma) | 0% | 0% |
| Fungal GR | 107% | 107% |

### Example 3: Use of the Glutathione Reductase to Reduce Cystine to Cysteine in Presence of Glutathione

Reduction of cystine to cysteine was carried out in presence of oxidized glutathione as mediator and NADPH as cofactor using fungal GR and yeast GR (sigma). The schematic reaction is as follow:

CYS−CYS+2GSH→2CYS+GSSG

GSSG+NADPH→2GSH+NADP+H

The reaction was performed in a 200 ml jacketed vessel with temperature and pH control. The total working volume was 100 ml. reactions were performed in two vessels. Vessel 1 contained the reference yeast GR (Sigma) and vessel 2 contained the thermostable fungal GR. Reactions were performed at 55 degrees Celsius and pH was controlled at 8 with NaOH (1M). Concentration of reactants and enzyme are summarized in Table 2.

TABLE 2
Concentrations of components applied in reduction of Cystine
using GR enzyme (in 100 mL)

| compounds | Vessel 1 | Vessel 2 |
|---|---|---|
| Enzyme | yeast GR (Sigma) | Fungal GR |
| Enzyme dosage (Units/ml)* | 2.0 | 2.0 |
| GSSG (g) | 0.1 | 0.1 |
| Cystine g) | 1.0 | 1.0 |
| NADPH (g) | 4.775 | 4.775 |

*units in Micro mol/milliliter/min.

Samples were taken at t=0 (just before addition of enzyme), 0.25, 1, 2, 3, 4, and 5 hours. 200 μl sample was centrifuged 1 minute at 13.000 RPM and 100 μl supernatant was transferred to 900 μl 0.111N HCL (final concentration of 0.1N HCL), mixed and stored at −20° C. All samples were analyzed by LCMS-MS Method to measure L-cystine, L-cysteine, GSH, GSSG and combinations of these components. In addition, the completeness of the reaction was visually confirmed by a clear sample solution, indicating that the low soluble L-cystine was reduced to the highly soluble L-cysteine. The cysteine formation in time is illustrated in FIG. 5. Complete reduction of ˜10 g/l L-cystine to L-cysteine with use of fungal GR within five hours of incubation. Level of cysteine in presence of yeast GR (sigma) did not increase in time showing that the enzyme was not active under applied condition.

1. A polypeptide having glutathione reductase activity selected from the group consisting of:
a) a polypeptide having an amino acid sequence comprising the mature polypeptide sequence of SEQ ID NO: 1;
b) a polypeptide comprising an amino acid sequence that has at least 50% sequence identity with the mature polypeptide sequence of SEQ ID NO: 1;
c) a polypeptide encoded by a nucleic acid comprising a sequence that hybridizes under medium stringency conditions to the complementary strand of the mature polypeptide encoding sequence of SEQ ID NO: 2; and
d) a polypeptide comprising an amino acid sequence encoded by a nucleic acid that has at least 50% sequence identity to the mature polypeptide coding sequence of SEQ ID NO: 2.

2. A polypeptide according to claim 1, which is derivable from Chaetomium thermophilum.

3. A nucleic acid encoding a polypeptide having glutathione reductase activity according to claim 1, which comprises a sequence that at least 50% sequence identity to the mature polypeptide encoding sequence of SEQ ID NO: 2.

4. An expression vector comprising a nucleic acid according to claim 3, operably linked to one or more control sequences that direct expression of the glutathione reductase in a host cell.

5. A recombinant host cell comprising a polypeptide according to claim 1, a nucleic acid encoding said polypeptide or an expression vector comprising said nucleic acid.

6. Recombinant host cell according to claim 5, which is a recombinant yeast cell, optionally a recombinant Saccharomyces cerevisiae cell.

7. A method for preparation of a polypeptide according to claim 1, which method comprises:
cultivating a suitable host cell in a suitable fermentation medium under conditions that allow for production of the polypeptide; and, optionally,
recovering the polypeptide.

8. A product comprising a polypeptide according to claim 1, for reduction of oxidized thiols.

9. A method for enzymatic reduction of cystine to cysteine, which method comprises:
contacting cystine with a reduction solution comprising a polypeptide according to claim 1, a cofactor and a mediator; and, optionally, recovering the cysteine.

10. Method according to claim 9, wherein contacting the cystine with the reduction solution is carried out at a temperature within a range of 25° C. to 75° C.

11. Method according to claim 10, wherein the mediator is glutathione.

12. Method according to claim 9, wherein the cofactor is nicotinamide adenine dinucleotide phosphate (NADPH), preferably in oxidized form (NADP+) or nicotinamide adenine dinucleotide (NADH), preferably in oxidized form (NAD+).

13. Method according to claim 9, wherein the reduction solution has a pH of at least 6.

14. A composition comprising cysteine and a polypeptide according to claim 1.

15. A composition according to claim 14, further comprising one or more selected from gluconic acid, formic acid, glucose, formate, nicotinamide adenine dinucleotide phosphate, nicotinamide adenine dinucleotide, glutathione, acetone and acetaldehyde.


SEQUENCE LISTING (2 sequences)

SEQ ID NO: 1 - protein, 469 aa, Chaetomium thermophilum
MAPISKETDFLVIGGGSGGLGAARAASGRYGARAMIIEGKRLGGTCVNVGCVPKKVTFNAAAIAETIHHSKAYGFNVQETAPFDWATFKAKRDAYVKRLNGIYERNLANDKVEYVHGWAKLISPNQVEVTLEDGSKTVVSAKKILIAVGGYPSPPPAIPGAEYGTNSDGFFDIDKLPKKVALVGAGYIAVEFAGMFNALGVETHLFIRHDHFLRAFDPMIQEGVTKEYERLGVKLHKRSALTRVDKDATTGQLTIHYKEGDGEGVIGDVDHLIWAIGRKPATQGLGLEAAGVKTDEKGYIVVDEYQNTSVENIYALGDVTGRVELTPVAIAAGRKLAARLYGPEQFRTAKLDYENVPSVVFAHPEVGAIGLTEPQAIEKYGKENVKVYKSNFIAMYYAMMEPEQKAPTSYKLVCVGPEERVVGLHIMGLGSAEILQGFGVAIKMGATKADFDNCVAIHPTSAEELVTMR

SEQ ID NO: 2 - DNA, 1410 bp, Artificial Sequence
  Codon Pair Optimized DNA Sequence
atggctccaatctccaaggaaactgacttcttggtcattggtggtggttctggtggtttgggtgccgctagagctgcttccggtcgttatggtgccagagctatgatcattgaaggtaagagattgggtggtacttgtgtcaacgttggttgtgttccaaagaaggtcactttcaacgctgctgccattgctgaaaccattcaccactccaaggcttacggtttcaacgttcaagaaaccgctccattcgactgggctactttcaaggccaagagagatgcttacgtcaagagattgaatggtatctacgaaagaaacttggctaacgacaaggttgaatacgtccacggttgggctaaattgatctctccaaaccaagttgaagtcactttagaagatggttccaagaccgttgtttctgccaagaagatcttgattgctgtcggtggttacccatctccaccaccagccattccaggtgctgaatacggtactaactccgatggtttcttcgacattgacaaattgcctaagaaggttgctttggtcggtgctggttacatcgccgttgaatttgctggtatgttcaacgccttgggtgtcgaaacccacttgttcatcagacacgaccatttcttgagagctttcgacccaatgatccaagaaggtgtcaccaaggaatacgaaagattaggtgtcaaattgcacaagagatctgctttgactagagttgacaaggatgctaccaccggtcaattaactattcactacaaggaaggtgatggtgaaggtgttatcggtgacgttgaccatttgatctgggctattggtagaaagccagctactcaaggtttaggtttggaagctgctggtgtcaagaccgatgaaaagggttacattgttgttgacgaataccaaaacacttctgttgaaaacatctacgctttgggtgatgtcaccggtcgtgtcgaattgaccccagttgccattgccgctggtagaaaattggctgctcgtctatacggtccagaacaattcagaaccgctaagttggactacgaaaacgttccatccgttgtctttgctcacccagaagttggtgctatcggtttgactgaacctcaagctatcgaaaagtacggtaaggaaaacgtcaaggtctacaaatctaacttcatcgccatgtactacgctatgatggaaccagaacaaaaggctccaacttcttacaaattggtttgtgttggtccagaagaaagagttgttggtttgcacatcatgggtttgggttctgctgaaatcttgcaaggtttcggtgtcgccatcaagatgggtgctactaaggctgatttcgacaactgtgttgccattcacccaacctctgctgaagaattagtcaccatgcgataa